(19) United States
(12) Patent Application Publication
(10) Pub. No.: US 2025/0242022 A1
(43) Pub. Date: Jul. 31, 2025

(54) ENHANCED CD8+ T CELLS FOR CANCER IMMUNOTHERAPY

(71) Applicant: HUMANITAS MIRASOLE S.P.A., Rozzano (MI) (IT)

(72) Inventors: Caterina SCIRGOLEA, Rozzano (MI) (IT); Enrico LUGLI, Rozzano (MI) (IT)

(73) Assignee: HUMANITAS MIRASOLE S.P.A., Rozzano (MI) (IT)

(21) Appl. No.: 18/866,915

(22) PCT Filed: May 19, 2023
(86) PCT No.: PCT/EP2023/063475

§ 371 Date: Nov. 18, 2024

(30) Foreign Application Priority Data
May 20, 2022 (IT) 102022000010535

(51) Int. Cl. A61K 40/11; A61K 40/15; A61K 40/31; A61P 35/00; C12N 5/0783

(52) U.S. Cl. CPC A61K 40/11; A61K 40/15; A61K 40/31; A61P 35/00; C12N 5/0636; C12N 2500/05; C12N 2501/2302

(57) ABSTRACT

The present invention refers to an in vitro or ex vivo method for the treatment of isolated CD8+ T cells. In particular, it refers to an in vitro or ex vivo method for obtaining an isolated population of CD8+ T cells comprising exposing isolated CD8+ T cells to an excess of sodium chloride. CD8+ T cells obtained by such method, pharmaceutical compositions including them and their medical uses, in particular for the prevention and/or the treatment of cancer, are also within the invention.

### FIELD OF THE INVENTION

The present invention refers to the field of cancer immunotherapy, in particular it refers to an in vitro or ex vivo treatment of CD8+ T cells to be used for cancer immunotherapy.

### BACKGROUND OF THE INVENTION

CD8+ T cells play a central role in tumor rejection but inevitably become dysfunctional or “exhausted” because of multiple mechanisms that concur in the TME, such as chronic antigen stimulation, immunosuppression and competition for limited resources (e.g., metabolites such as glucose) (McLane et al., 2019). T cell-mediated immunity in the tumor microenvironment (TME), either in response to checkpoint blockade immunotherapies (Siddiqui et al., 2019) or following Adoptive Cell Therapy (ACT) are thought to be largely dependent on precursor T cells with stem-like potential.

Stem-like T cells in humans (Gattinoni et al., 2011), more recently in tumors (Brummelman et al., 2018) have been identified; also, those molecular characteristics that are involved in functional vs. dysfunctional fate decision in these stem-like precursors have been identified (Galletti et al., 2020). Notably, simultaneous expression of stem-like and effector genes in highly functional stem-like precursors (hereafter referred to as the stem/effector-hybrid), resulted in enhanced anti-tumor immunity upon ACT.

ACT has the advantage over other immunization strategies in that it does not require in vivo activation of antigen specific T cells by patients with a compromised immune system.

However, there is still not a definitive and optimal protocol for ex vivo T cell activation to promote a favorable anti-tumor response.

Recent data have shown that increased K+ in the TME could contribute to T cell dysfunction (Eil et al., 2016), shifting the attention to ionic imbalance in the TME.

Jorg et al. investigates the effect of NaCl on Th17 cells.

In WO2021222479 methods of culturing immune cells or T memory stem cells comprising also stimulation of T cells in hypertonic conditions are disclosed.

Therefore, there is still the need of a method for obtaining activated T cells, in particular CD8+ T cells, which can be advantageously used in cancer immunotherapy.

### SUMMARY OF THE INVENTION

The inventors have now found that NaCl supplementation augments T cell effector functions and tumor control in vitro and in vivo, possibly by acting as a potent inducer of the stem/effector hybrid CD8+ T cell state. In particular, it was observed an increased production of IFN-γ and expression of the degranulation marker CD107a when CD8+ T cells are grown in hypertonic conditions.

More importantly, it was found that CD8+ T cells cultured in an excess of sodium chloride comprised between 160 and 250 mM, in particular comprised between 163.4-183.4 mM, are able to reduce tumor growth in mice injected with human melanoma cells and to increase in vitro lysis of target human melanoma cells.

It is therefore an object of the invention an in vitro or ex vivo method for obtaining an isolated population of CD8+ T cells comprising exposing isolated CD8+ T cells to an excess of sodium chloride by culturing said cells in a medium containing sodium chloride in a concentration comprised between 160 and 250 mM.

For sodium chloride in excess it is intended in an amount higher than the amount commonly used in CD8+ T cell cultures. The amount commonly used in CD8+ T cell cultures is for example equal or less than 100 mM or 103.4 mM.

Preferably the concentration of sodium chloride to which the CD8+ T cells are exposed is comprised between 160 and 180 mM, even more preferably it is about 180 mM or 163.4 mM or 183.4 mM.

Preferably the CD8+ T cells are grown in a medium already containing NaCl and an excess of NaCl is added to reach the desired concentration. For example, the medium comprises 100 mM of NaCl or 103.4 mM NaCl and 80 mM of NaCl are further added.

Preferably, the CD8+ T cells are exposed to an excess of sodium chloride for a period of between 3 and 10 days, preferably for 5 days.

Preferably, said isolated CD8+ T cells have been previously isolated from a subject, for example from a blood sample obtained from a subject. Preferably, said subject is a human subject.

Preferably the method further comprises activation and/or expansion of the CD8+ T cells.

Preferably, the CD8+T cells are also exposed, before, simultaneously or after exposure to NaCl, to other compounds, for example known stimulators of activation and/or expansion of T cells, such as for example, cytokines, such as IL-2, anti-CD3 and anti-CD28 antibodies, preferably conjugated to beads.

In an embodiment, the method comprises the following steps:
a) plating CD8+T cells previously isolated from a subject in a suitable plate;
b) adding to the plated cells a suitable medium comprising a concentration of sodium chloride comprised between 160 and 250 mM.

The CD8+T cells obtained with the method of the invention are characterized by enhanced effector function, and an increased proliferative potential when infused in vivo.

The isolated population of CD8+ T cells obtained with the method of the invention is also an object of the invention.

A pharmaceutical composition comprising such isolated population of CD8+ T cells is also an object of the invention.

It is also an object of the invention the isolated population of CD8+ T cells obtained with the method of the invention or the pharmaceutical composition comprising such isolated population of CD8+ T cells for use as a medicament, in particular for use for the prevention and/or treatment of cancer.

More preferably, the isolated population of CD8+ T cells obtained with the method of the invention or the pharmaceutical composition comprising such isolated population of CD8+ T cells is for use for cancer immunotherapy.

In an embodiment the isolated population of CD8+ T cells obtained with the method of the invention is for use in an adoptive cell therapy (ACT).

Preferably, the CD8+ T cells for use for the prevention and/or treatment of cancer are allogeneic or autologous to the treated subject. Preferably, the cells are autologous to the subject.

### DETAILED DESCRIPTION OF THE INVENTION

### Figures

FIG. 1. Stimulation of naive CD8+ T cells with NaCl. A. Human naïve CD8+ T cells stimulated with IL-2+aCD3/CD28 for 5 days with different mM concentrations of NaCl and the equiosmolar urea. Cells were analyzed by flow cytometry to determine cell viability. B Expression of GZMB represented as integrated Median Fluorescent Intensity (% positive cells×MFI of positive cells). C. IFN-γ production by naïve CD8+ T cells stimulated for 5 days with anti-CD3/CD28 and IL-2 plus NaCl or urea following restimulation with PMA/ionomycin for 3 hours. One-way ANOVA with Tukey post-hoc test. N=4, 2 independent experiment; ****P<0.0001, ***P=0.0002, **P=0.0021, *P=0.0332.

FIG. 2. CD8+ T cells mediate anti-tumor response in HSD treated mice. A. Experimental design. C57BL/6 mice were kept on normal diet (ND) or were fed a high salt diet (HSD) for 14 days before tumor inoculation. Mice were intraperitoneally injected with anti-CD8 or IgG isotype control monoclonal antibodies the day before tumor inoculation and then twice a week. After tumor challenge the mice were further kept on the same diets until sacrifice. B. Mice pre-fed on the respective diets were challenged with MC38 colon adenocarcinoma cells by subcutaneous (s.c.) injection. Growth curve shows tumor volume as mean±S.E.M. (n=8 per group) at the indicated time points from one experiment. Statistical analysis was performed by two-way ANOVA with Tukey post-hoc test; ****P<0.0001, ***P=0.0002, **P=0.0021, *P=0.0332. ND, normal diet; HSD, high salt diet.

FIG. 3. NaCl reprograms CD8+ T cells. A. Representative flow cytometric plot showing the effect of 80 mM NaCl and equiosmoloare urea (176 mM) on CCR7, CD45RO, B. GZMB and TIM3 expression. Numbers indicate the percentage of positive cells identified by the gates. Similar data were obtained from 5 more healthy donors in 2 independent experiments. C. UMAP analysis of concatenated CD8+ T cells (1,500 cells/sample) from the PBMCs of 3 healthy donors. Top: PhenoGrah clusters. Bottom: cell distribution according to NaCl or urea treatment. D. Balloon plot map showing the percent positive cells and the median fluorescence intensity (MFI) of specific markers (columns) in discrete PhenoGraph clusters (rows) as identified from the analysis of CD8+ naïve T cells from 3 healthy donors stimulated for 5 days with anti-CD3/CD28 and IL-2 plus 80 mM NaCl or 176 mM urea. Hierarchical metaclustering grouped markers and clusters with similar immunophenotypes. Bar plots show the percent frequency of each cluster after NaCl or Urea treatment. T_{N}, T naïve; T_{CM}, T central memory; T_{EM}, T effector memory. E. Flow cytometry plot from a representative healthy donor showing the production of CD107a and IFN-γ by CD8+ T_{N} cells obtained as in A and restimulated with PMA/ionomycin. Numbers indicate the percentage of positive cells identified by the gates. Similar data were obtained from 5 more healthy donors in 2 independent experiments. F. Representative flow cytometric analysis of 2-NBDG and Bodipy FL C16 uptake by CD8+ T cells treated with IL-2+aCD3/CD28 plus or minus NaCl 80 mM. N=2, one experiment.

FIG. 4. NaCl generates CD8+ T cells with increased proliferative potential in vivo. A. Experimental design. NSG mice were retro-orbitally injected with CD8+ naïve T cells stimulated for 5 days with IL-2 plus anti-CD3/CD28 in the absence or presence of 60 mM NaCl. B. Absolute numbers of CD8+ T cells in peripheral blood on days 14, 21 and 28 after transfer. Data are representative of one experiment (n=3 per condition). Similar data were obtained in two additional independent experiments. Bars indicate mean±S.E.M. Two-way ANOVA with Bonferroni post-hoc test. ****P<0.0001.

FIG. 5. Tumor growth curves in immunodeficient NSG mice injected with human melanoma cells (624.38) and adoptively transferred 7 days later with MART-1 specific CD8+ T cells expanded in vitro in RPMI 1640 culture medium containing 103.4 mM NaCl or 163.4 mM NaCl. Two-way ANOVA, n=6. ACT: adoptive cell transfer

FIG. 6. Lysis of target human melanoma cells (624.38) by CD8+ T cells stimulated with interleukin (IL)-2 alone or IL-2 plus aCD3/CD28 in RPMI 1640 culture medium containing 103.4 mM NaCl or 183.4 mM NaCl at different effector-to-target (E:T) ratios. Two-way ANOVA, n=2. P-values refer to the IL-2+aCD3/28 (103.4 mM NaCl) vs IL-2+aCD3/28 (183.4 mM NaCl) comparison.

FIG. 7. Magnetically-enriched human naïve CD8+ T cells from healthy donors were stimulated for 5 days with recombinant IL-2 alone or plus anti-CD3/CD28 Dynabeads in RPMI1640 culture medium containing 103.4 mM NaCl or an extra 20 mM, 40 mM or 80 mM NaCl. Curves represent the percentage of live CD8+ T cells at day 5 of culture. N=2.

For “CD8+ T cells” is herein intended T cells characterized by the presence on the cell surface of the protein CD8. CD8+ T cells are also known in the field as “killer T cells”.

“Sodium chloride” and “NaCl” are herein used as synonym.

For “about” is intended the indicated value+/−1%.

The method of the invention is performed in vitro or ex vivo. For ex vivo is intended that it is performed on cells isolated from a subject.

According to the method of the invention isolated CD8+ T cells are exposed to an excess of sodium chloride.

For exposed it is intended that CD8+ T cells are placed in contact with sodium chloride.

According to the method of the invention, CD8+ T cells are grown in a cell medium comprising sodium chloride in excess, i.e. higher with respect to concentrations commonly used in CD8+ T cell cultures, wherein the cell medium comprises a concentration of sodium chloride comprised between 160 and 250 mM, preferably between 160 and 180 mM, even more preferably it is about 180 mM. In some embodiments, the concentration of sodium chloride in said cell medium is comprised between 160 and 190 or between 160 and 170 mM or between 170 and 180 mM or between 180 and 190 mM. In some preferred embodiments, said concentration is comprised between 163.4 and 183.4 mM or it is 160 mM, 163.4 mM, 170 mM, 180 mM or 183.4 mM.

In some embodiments, said concentration is reached by adding further sodium chloride to a commonly used cell medium which may already contain sodium chloride. Said cell medium is for example RPMI 1640, which may contain NaCl in a concentration of about 103.4 mM.

The cell medium can further comprise suitable components and elements which are commonly used for the growth of T cells, especially of CD8+ T cells, according to the general knowledge in the field. For example it can comprise fetal bovine serum (FBS), antibiotics, such as penicillin-streptomycin, and/or amino acids, such as L-glutamine.

In an embodiment, the medium is RPMI 1640 medium supplemented with 10% FBS, 1% penicillin-streptomycin and 1% L-glutamine

CD8+ T cells can be exposed to an excess of sodium chloride for a suitable time which can be chosen by the skilled person. Preferably, the CD8+ T cells are cultivated in the medium comprising an excess of sodium chloride according to the present invention for a period of between 3 and 10 days, preferably for 5 days.

CD8+ T cells can be previously isolated from a subject, such as a human or animal subject, according to common methods known in the art. For example they can be isolated from the blood obtained by a blood draw from a subject.

CD8+ T cells can be from an animal or from a human subject, preferably they are human.

In an embodiment, CD8+ T cells are isolated from peripheral blood mononuclear cells (PBMC) by standard procedures.

In an embodiment, the method of the invention further comprises activation and/or expansion of the CD8+ T cells. Activation and expansion can occur according to methods known in the art. For example, the cells can be exposed to non-specific T cell stimuli and/or one or more cytokines. Exemplary non-specific T cell stimuli include, anti-CD3 antibodies and anti-CD28 antibodies. In preferred embodiment, the non-specific T cell stimulus may be anti-CD3 antibodies and anti-CD28 antibodies conjugated to beads. Any one or more cytokines may be used. Exemplary cytokines include interleukin IL-2, IL-7, IL-21, and IL-15, preferably IL-2 is used. Such cytokines and/or antibodies can be added to the cell medium.

In an embodiment, CD8+ T cells are stimulated by culturing said cells in a cell medium comprising IL-2 and/or anti-CD3 and anti-CD28 antibodies conjugated to beads, preferably in a bead-to-cell ratio 1:2. A preferred concentration of IL-2 is between 5 and 20 U/mL, preferably it is 5 U/mL.

In an embodiment, the number of T-cells can be further expanded in vitro by suitable methods known in the art, such as cultivating the T cells in presence of suitable stimuli.

In an embodiment, the method of the invention comprises the following steps:
a) plating CD8+T cells previously isolated from a subject in a suitable plate;
b) adding to the plated cells a suitable medium comprising a concentration of sodium chloride comprised between 160 and 250 mM, preferably between 160 and 180 mM.

Preferably, the CD8+ T cells are cultivated in the medium for a period of between 3 and 10 days, preferably for 5 days.

Optionally in step b) further compounds are added to the medium, such as known stimulators of activation and/or expansion of T cells. Exemplary activators are, cytokines, such as IL-2, and anti-CD3 and anti-CD28 antibodies, preferably conjugated to beads.

Any medium suitable for culturing T cells can be used. For example, RPMI 1640 culture medium, which is commercially available, can be used.

In an embodiment, the isolated CD8+ T cells are antigen-specific, i.e. they are modified and/or selected for recognizing a specific antigen, such as a tumoral antigen.

For example, said cells can be obtained according to the following protocol:
PBMCs are previously isolated from a subject and CD14+, CD8+ and CD4+T cells are enriched for example by magnetic column separation;
CD14+ cells are cultured in suitable medium such as RPMI 1640 and stimulated with 800 U/ml IL-4 plus 1000 U/ml GM-CSF for 5 days to generate mature dendritic cells;
mature dendritic cells are loaded with a specific antigen, such as Mart-1 26-35 peptide, preferably plus 3 μg/ml β2 microglobulin and 10 μM of tetanus toxoid for 3 hours, then cultured, for example in a 1:6 ratio, with CD8+ T cells and irradiated CD4+ T cells;
IL-2 (e.g. 20 U/ml) are added to CD8+ T cells, for example starting from day 3 and replaced every 2-3 days;
CD8+ T cells are restimulated, for example at day 14, with the antigen, for example with Mart-1 26-35 peptide-pulsed autologous monocytes;
culture medium, such as RPMI1640, containing an excess of NaCl according to the method of the invention is added, for example at day 16.

The isolated population of CD8+T cells obtained with the method of the invention is a further object of the invention.

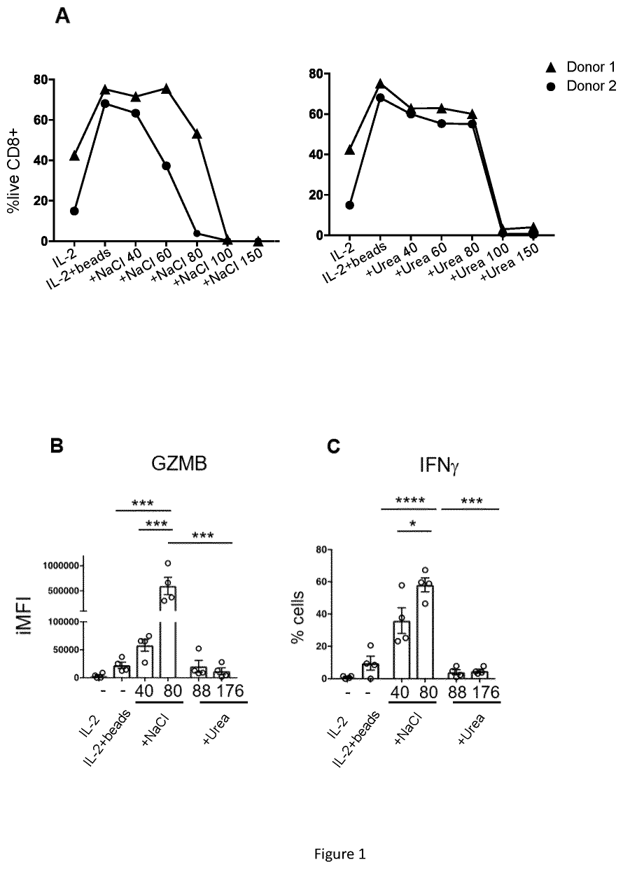

Preferably, CD8+T cells obtained with the method of the invention are characterized by:
an effector phenotype characterized by the expression of CD45RO and lack of expression of CCR7 (see for example FIG. 3A);
an higher expression of IFNγ as compared to control CD8+ T cells stimulated with urea (see for example FIG. 1C); and
an higher expression of marker CD107a as compared to control CD8+ T cells stimulated with urea (see for example FIG. 3E).

For “control CD8+ T cells” is intended isolated CD8+ T cells cultivated in a medium which does not comprise NaCl in a concentration equal to or higher than 160 mM.

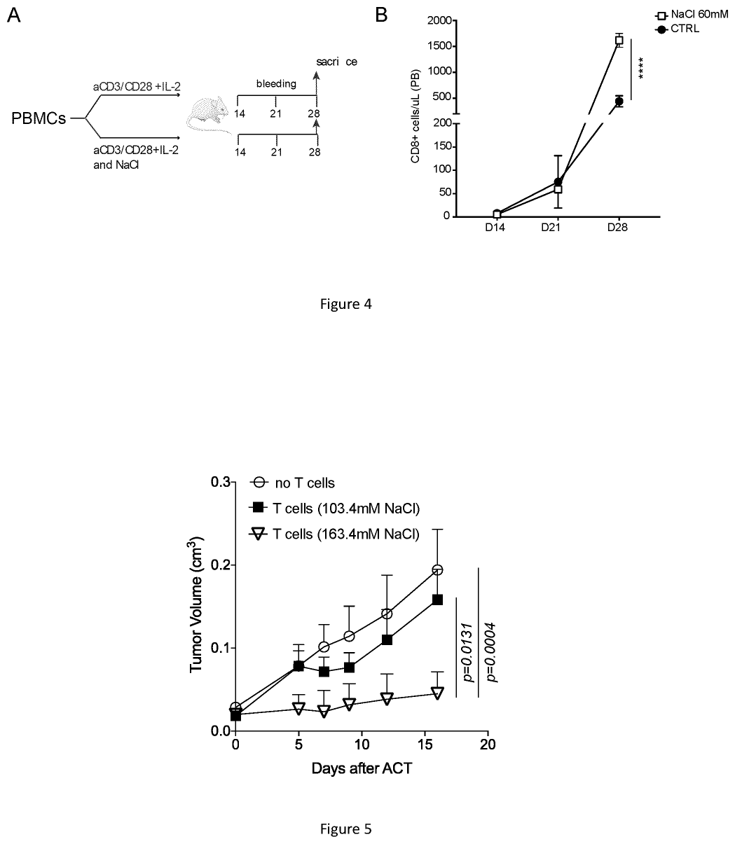

Furthermore, such cells when tested in vitro against a tumoral cell line are more able of inducing lysis of target tumoral cells with respect to control (see for example FIG. 4). Also, when administered to immunodeficient mice such cells show a higher anti-tumor growth activity with respect to CD8+ T cells not exposed to an excess of sodium chloride (see for example FIG. 5).

The CD8+T cells obtained with the method of the invention can be included in a composition, such as a pharmaceutical composition. In this regard, the invention provides also a pharmaceutical composition comprising CD8+T cells obtained with the method of the invention and at least one pharmaceutically acceptable carrier and/or vehicle.

The carrier and vehicle can be any of those conventionally used for the administration of cells. Such pharmaceutically acceptable carriers and vehicle are well-known to those skilled in the art and are commercially available.

The CD8+ T cells or the pharmaceutical composition of the invention may be administered in any suitable manner. In an embodiment they are administered as an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Methods for preparing administrable (e.g., parenterally administrable) compositions are known or apparent to those skilled in the art and are described in more detail in, for example, Remington's Pharmaceutical Science (17th ed., Mack Publishing Company, Easton, PA, 1985).

The dose of the CD8+ T cells to be administered, for example the number of cells, is an amount sufficient to obtain the desired effect, in particular the prevention or the treatment or the control of cancer. The physician can determine a suitable amount depending on the severity of the disease, the conditions of the patient and other suitable parameters according to the general knowledge in the field.

The invention also comprises the isolated population of CD8+ T cells obtained with the method of the invention for use for the prevention and/or treatment of cancer.

According to the present invention, for “prevention” is intended that administration of the agent decreases the chance of developing a disease or condition, i.e. it decreases the chance of developing a cancer. In some embodiments, for “prevention” is intended that administration of the agent stops or slows down progression of a disease that has already begun. For example, in some embodiments the agent of the invention is administered to a subject who already has cancer and the cancer does not develop to a more advanced stage.

According to the present invention, for “treatment” it is intended that administration of the agent improves or cures or reverts a condition or a disease, i.e. it improves or cures or reverts a cancer. In some embodiments, for “treatment” it is intended that the disease, such as cancer, is not completely cured but it reverts to a less advanced stage. Any amount of any level of treatment or prevention of cancer can be provided by administration of the cells of the invention. Furthermore, the treatment or prevention provided by the invention can include treatment or prevention of one or more conditions or symptoms of the disease, e.g., cancer, being treated or prevented.

In an embodiment of the invention, the CD8+ T cells obtained with the method of the invention are for use in cancer adoptive cell therapy.

For “adoptive cell therapy” it is intended a type of immunotherapy in which T cells are administered to a patient to help the body fight a disease, such as cancer. In cancer adoptive cell therapy, T cells are usually isolated from the patient's own blood or tumor tissue, grown in laboratories, and then administered back to the patient to help the immune system fight the cancer. Adoptive cell transfer, cellular adoptive immunotherapy, and T-cell transfer therapy are synonymous.

In an embodiment, an adoptive cell therapy according to the invention comprises:
exposing CD8+ T cells previously isolated from a subject to an excess of sodium chloride according to the method of the invention;
administering the obtained isolated CD8+ T cell population to a subject in need thereof, wherein preferably said subject is the same subject from which CD8+ T cells were previously isolated.

The cells administered to a subject in need thereof can be cells that are allogeneic or autologous to the host. Preferably, the cells are autologous to the host. In an embodiment, the CD8+ T cells are previously isolated from a subject affected by a cancer, then exposed to NaCl according to the method of the invention and then administered again to the same subject.

The isolated CD8+ T cells of the method of the invention can be further modified, before or after exposure to an excess of sodium chloride according to the method of the invention. For example, they can be genetically engineered to enhance their cancer-fighting capabilities and/or to increase their ability to specifically target an antigen of interest, such as a tumoral antigen. For example said isolated CD8+ T cells can be modified to express an antigen receptor, such as an antibody, a single-chain variable fragment (scFv), a T Cell receptor (TCR) or a chimeric antigen receptor (CAR). Such modified cells can be advantageously used in adoptive cell therapies such as: Tumor-Infiltrating Lymphocyte (TIL) Therapy, Engineered T Cell Receptor (TCR) Therapy, Chimeric Antigen Receptor (CAR) T Cell Therapy and Natural Killer (NK) Cell Therapy.

The cancer can be any cancer, including any of sarcomas (e.g., synovial sarcoma, osteogenic sarcoma, leiomyosarcoma uteri, and alveolar rhabdomyosarcoma), lymphomas (e.g., Hodgkin lymphoma and non-Hodgkin lymphoma), hepatocellular carcinoma, glioma, head-neck cancer, acute lymphocytic cancer, acute myeloid leukemia, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer (e.g., colon carcinoma), esophageal cancer, cervical cancer, gastrointestinal carcinoid tumor, hypopharynx cancer, larynx cancer, liver cancer, lung cancer, malignant mesothelioma, melanoma, multiple myeloma, nasopharynx cancer, ovarian cancer, pancreatic cancer, peritoneum, omentum, and mesentery cancer, pharynx cancer, prostate cancer, rectal cancer, renal cancer, small intestine cancer, soft tissue cancer, stomach cancer, testicular cancer, thyroid cancer, ureter cancer, and urinary bladder cancer. In a preferred embodiment, it is melanoma.

Further active ingredients can be administered to the subject together with the CD8+ T cells of the invention. Preferably, such further active ingredient is a chemotherapeutic agent. A “chemotherapeutic agent” is a chemical compound useful in the treatment of cancer. Any chemotherapeutic agent known in the field can be used, in particular agents which are known to be effective in the cancer to be treated or which are known to enhance activity of T cells.

When the cells of the invention are administered with one or more additional therapeutic agents, one or more additional therapeutic agents can be coadministered to the mammal. By “coadministering” is meant administering one or more additional therapeutic agents and the cells of the invention sufficiently close in time such that the cells of the invention can enhance the effect of one or more additional therapeutic agents. In this regard, the cells of the invention can be administered first and the one or more additional therapeutic agents can be administered second, or vice versa. Alternatively, the cells of the invention and the one or more additional therapeutic agents can be administered simultaneously.

The invention will be now described by means of illustrative examples.

### EXAMPLES

### Example 1

Protocol of Ex-Vivo Activation of Human CD8+ T Cells with NaCl

### Reagents:

1. NaCl MERK Cat. 7647-14-5
2. Urea Bio-Rad Cat. 161-0731
3. RPMI 1640 medium supplemented with 10% FBS, 1% penicillin-streptomycin and 1% L-glutamine
4. 96 well plate, U-bottom
5. Recombinant human Interleukin 2, Miltenyi Biotec
6. Dynabeads® Human T-Activator CD3/CD28, LifeTechnologies, Cat. 11132D
7. EasySep human Naïve CD8+ T Cell Isolation Kit, StemCell Technologies, Cat. 17968
8. Advanced Osmometer Model 3250—Advanced Instrument Inc.

We isolated human naïve CD8+T cells from frozen healthy donors PBMCs using the EasySep human Naïve CD8+ T Cell Isolation Kit (StemCell Technologies). Enriched Naïve CD8+ T cells were plated in 96 wells plate U-bottom (1×10⁵ per well) and left at 37° C. for 3 hours. To investigate the effect of NaCl on the in vitro differentiation of human CD8+ cells, we stimulated T cells with 5 U/ml IL-2 (1 ng/ml) plus anti-CD3 and CD28 Dynabeads (bead-to-cell ratio 1:2) in RPMI 1640 containing an excess of 40-150 mM NaCl, thus mimicking concentrations that could be found in the interstitium of animals fed a high-salt diet (MacHnik et al., 2009) (FIG. 1). We used equiosmolar urea, an osmolyte able to pass through cell membranes, as an osmotic control. We measured the osmolarity of the cell medium using the Advanced Osmometer Model 3250 (Advanced Instrument Inc.). Note that the final concentration of NaCl and urea are different to match equiosmolarity.

Since we observed an increased mortality above 80 mM (FIG. 1A) we maintained the NaCl treatment conditions at Δ80 mM (with a final concentration of up to 180 mM, considering that RPMI 1640 contains 100 mM NaCl). We stimulated naïve CD8+ T cells with or without NaCl for 5 days and analyzed the phenotype by flow cytometry. We observed that the effect of NaCl was dose dependent and the highest production of Granzyme B (GZMB) and Interferon-gamma (IFN-γ) could be achieved with 80 mM NaCl. Equiosmolar urea did not increase these parameters compared to untreated control (FIG. 1B,C).

### Example 2

### NaCl Reduces Tumor Growth by Regulating the CD8+ T Cell Response

Recent evidence has shown that administration of high-salt diet (HSD) to two allograft murine tumor models (melanoma B16F10 and mammary cancer 4T1) significantly reduced tumor growth by modulating MDSCs differentiation and by promoting tumor T cell infiltration (He et al., 2020).

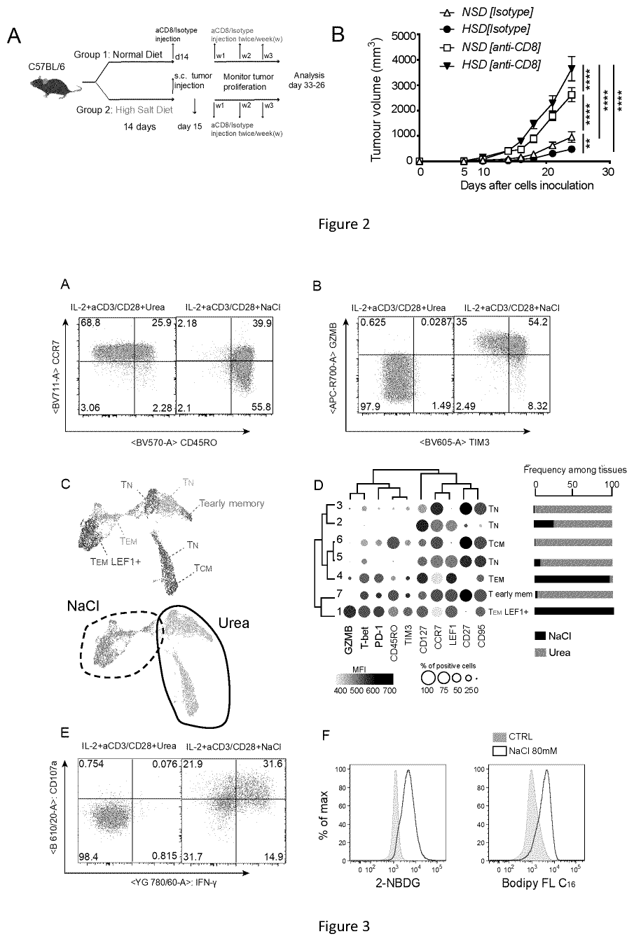

It is currently unclear whether T cells are directly involved in tumor rejection after HSD administration. To test this, we randomized C57BL/6 mice to receive normal diet (ND) or HSD for 2 weeks followed by the injection of MC38 tumor cells (FIG. 2A). Mice receiving HSD showed a reduced tumor growth compared to mice receiving ND (FIG. 2B). HSD favors the infiltration of T cells with effector/cytotoxic phenotype in experimental tumors, but their function in controlling tumor growth is unknown (He et al., 2020).

We then asked whether CD8+ T cells were directly involved in restraining tumor proliferation. To this aim, we depleted CD8+ T cells using an anti-CD8 monoclonal antibody in mice fed with ND or HSD (FIG. 2B). Mice receiving ND failed to control MC38 colon cancer cell growth when CD8+ T cells were depleted. The effect of HSD was lost in the absence of CD8+ T cells, (FIG. 2B), thereby demonstrating that HSD-induced anti-tumor activity is largely mediated by CD8+ T cells.

### Example 3

### NaCl Primes CD8+ T Cells for Effector Functions

Since we observed a central role of CD8+ T cells in regulating anti-tumor response in mice treated with HSD, we wanted to understand the role of NaCl on human CD8+ T cell differentiation and metabolism. CD8+ T cells in the TME have defective immune activation due to limited exposure to effector cytokines such as IL-2 (Bukowski et al., 1998; Rayman et al., 2000).

To mimic this scenario, we activated human naïve CD8+ T cells for 5 days with suboptimal concentrations of IL-2 in the presence or absence of NaCl. Urea was used as an osmotic control.

We observed that NaCl induces CD8+ effector differentiation (CD45RO+CCR7−) compared to the urea-treated cells that maintain mostly a naïve (CCR7+CD45RO−) or central memory (CCR7+CD45RO+) phenotype (FIG. 3A).

Moreover, we noticed increased Granzyme B (GZMB) and TIM3 expression (FIG. 3B), suggesting that NaCl induces a more activated/cytotoxic phenotype in CD8+ T cells.

To investigate more in detail the phenotype of NaCl-treated cells, we designed a multidimensional flow cytometry panel including markers informative of CD8+ T cell differentiation and activation. We used this panel to investigate the activation and functional status of naïve CD8+ T cells from 3 healthy donors stimulated with anti-CD3/CD28 plus IL-2 (suboptimal 5 U/mL (1 ng/mL) concentration) in the presence of NaCl or urea. By applying the unsupervised clustering algorithm Phenograph, we identified 7 different clusters of CD8+ T cells (FIG. 3C,D). In line with our previous results in vitro, we observed that effector memory CD8+ T cells (T_{EM}) represented by C1 and C4 were enriched after NaCl treatment (FIG. 3C,D). Interestingly, C1 overexpressed PD-1, TIM3 and GZMB, along with LEF1, a transcription factor involved in the long-term persistence of stem-like memory T cells. Differently, naïve T cells (T_{N}, C2, C3, C5), central memory T cells (T_{CM}, C6) and early memory T cells (C7) were enriched upon urea treatment. We next investigated the functional capacity of NaCl-treated cells following pharmacological stimulation with phorbol 12-myristate 13-acetate (PMA) and ionomycin by measuring effector molecules production by flow cytometry. In line with the acquisition of several markers related to immune activation and effector differentiation, we observed an increased production of IFN-γ and expression of the degranulation marker CD107a in cells cultured with NaCl in vitro (FIG. 3E). In line with the increased effector differentiation, T cells cultured in the presence of NaCl were characterized by major glucose utilization from the extracellular milieu, as revealed by the increased uptake of the fluorescent glucose analog 2-NBDG, and by increased extracellular-derived fatty acid (FA) utilization, required to fuel oxidative phosphorylation (FIG. 3F).

### Example 4

### NaCl-Treated CD8+ T Cells Show Increased Proliferation In Vivo

NaCl-treated cells upregulate memory-related genes but at the same time they express exhaustion-related markers such as PD-1 and TIM3 (FIG. 3B-D). This raises the question if these cells, although undergoing effector differentiation, maintain memory features such as the long-term persistence in vivo.

To further clarity this aspect, we monitored the proliferation capacity of CD8+ T_{N} cells activated in the presence or absence of NaCl for 5 days following adoptive transfer in NOD.Cg-Prkdc^{scid} IL2rg^{tm1Wjf}/SzJ (NSG) mice (FIG. 4A, B). In the blood of these mice, we observed increased expansion of cells previously cultured with NaCl.

### Example 5

### Materials and Methods

### Mice and Tumor Model

7-week old NOD.Cg-Prkdcscid II2rgtm1WjI/SzJ (NSG) female mice (Jackson Laboratories) were subcutaneously injected with 1×106 624-38 melanoma cells in the right flank. After one week, tumors became palpable, were randomized in the different groups and were injected i.v. with 8×106 MART-1 specific CD8+ T cells (200 μl in PBS final volume). Tumor growth was monitored by measuring the two visible dimensions with a caliper every 2-4 days.

### Human Antigen-Specific CD8+ T Cell Expansion and Adoptive Transfer in Immunodeficient Mice

PBMCs were isolated from a healthy HLA-A*02:01 donor by standard Ficoll procedure and CD14+, CD8+ and CD4+ T cells were enriched by magnetic column separation (Miltenyi Biotec). CD14+ cells were cultured in RPMI 1640 and stimulated with 800 U/ml IL-4 (Proleukin, Novartis) plus 1000 U/ml GM-CSF (Miltenyi) for 5 days to generate mature dendritic cells. Mature dendritic cells were loaded with Mart-126-35 peptide (lba Lifesciences) plus 3 μg/ml β2 microglobulin (Lee Biosolutions) and tetanus toxoid (10 μM; Pepscan) for 3 hours, then cultured in a 1:6 ratio with CD8+ T cells and irradiated CD4+T cells. IL-2 (20 U/ml; Proleukin, Novartis) was added to CD8+ T cells starting from day 3 and replaced every 2-3 days. CD8+ T cells were restimulated at day 14 with Mart-126-35 peptide-pulsed autologous monocytes and at day 16 either RPMI1640 culture medium containing 103.4 mM NaCl or 163.4 mM NaCl was added. Cells were transferred in mice 5 days later.

### Results

Immunodeficient NSG mice injected with human melanoma cells were adoptively transferred 7 days later with MART-1 specific CD8+ T cells expanded in vitro in RPMI 1640 culture medium containing 103.4 mM NaCl or 163.4 mM NaCl.

As shown in FIG. 5, cells expanded in vitro in a medium containing 163.4 mM NaCl are more able to control tumor growth with respect to cells expanded in 103.4 mM NaCl concentration.

### Example 6

### Materials and Methods

### Cytotoxicity Assay of CD8+ T Cells Towards 624.38 Melanoma Cells

Effector CD8+ T cells were obtained from magnetically-enriched human naïve CD8+ T cells (EasySep Human Naïve CD8+ T cell isolation kit, Stem Cell Technologies) and stimulated for 5 days with recombinant IL-2 (5 U/ml; Miltenyi) plus anti-CD3/CD28 Dynabeads (ThermoFisher Scientific) in RPMI1640 culture medium containing 103.4 mM NaCl or 183.4 mM NaCl.

Target cells (624.38 melanoma cells) were stained with Cell Trace Violet dye, washed, resuspended in complete RPMI 1640 and incubated overnight in a flat-bottom 96-well plate. The day after, target cells were cultured with effector CD8+ T cells at 10:1 and 5:1 effector:target ratios for 4 hours at 37° C. After incubation, cells were stained with Zombie AQUA (BioLegend) and anti-human CD8 antibody (BD Biosciences) directly in the cell plate for 30 minutes at 37° C. Following staining, cells were collected and stained with Helix NP™ NIR and Annexin V in binding buffer (BioLegend).

Specific lysis was calculated according to the following formula:

$$1 - ( \frac{\% viable target cells after coculture with effector cells}{\% viable target cells alone} ) \times 100$$

### Results

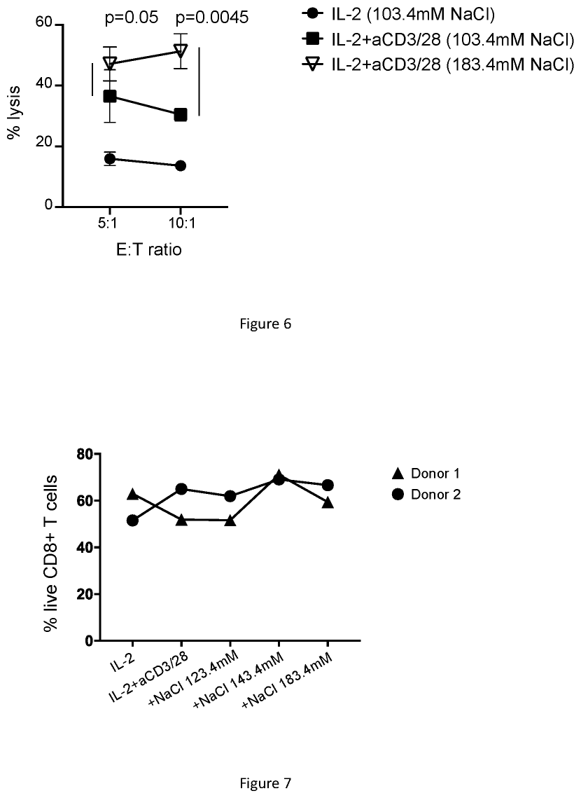

As shown in FIG. 6, CD8+ T cells expanded in vitro with 183.4 mM NaCl show a higher ability to induce lysis of target tumoral cells with respect to cells expanded in a medium with 103.4 mM NaCl concentration.

### Example 7

### Materials and Methods

Stimulation of Human Naive CD8+ T Cells In Vitro with NaCl

Magnetically-enriched human naïve CD8+ T cells (EasySep Human Naïve CD8+ T cell isolation kit, Stem Cell Technologies) from healthy donors were stimulated for 5 days with recombinant IL-2 (5 U/ml; Miltenyi) plus anti-CD3/CD28 Dynabeads (ThermoFisher Scientific) in RPMI1640 culture medium containing 103.4 mM NaCl or an extra 20 mM, 40 mM or 80 mM NaCl. After 5 days, cells were collected and stained with Zombie AQUA (BioLegend) to assess the viability by FACS.

### Results

As shown in FIG. 7, the addition to the medium of an excess of NaCl up to 80 mM does not affect cell viability.

### REFERENCES

MacHnik, A., Neuhofer, W., Jantsch, J., Dahlmann, A., Tammela, T., MacHura, K., Park, J. K., Beck, F. X., Müller, D. N., Derer, W., et al. (2009). Macrophages regulate salt-dependent volume and blood pressure by a vascular endothelial growth factor-C-dependent buffering mechanism. Nat Med 15, 545-552.
Brummelman, J., Mazza, E. M. C., Alvisi, G., Colombo, F. S., Grilli, A., Mikulak, J., Mavilio, D., Alloisio, M., Ferrari, F., Lopci, E., et al. (2018). High-dimensional single cell analysis identifies stem-like cytotoxic CD8+ T cells infiltrating human tumors. J Exp Med 215, 2520-2535.
Bukowski, R. M., Rayman, P., Uzzo, R., Bloom, T., Sandstrom, K., Peereboom, D., Olencki, T., Budd, G. T., McLain, D., Elson, P., et al. (1998). Signal transduction abnormalities in T lymphocytes from patients with advanced renal carcinoma: Clinical relevance and effects of cytokine therapy. Clinical Cancer Research 4, 2337-2347.
Eil, R., Vodnala, S. K., Clever, D., Klebanoff, C. A., Sukumar, M., Pan, J. H., Palmer, D. C., Gros, A., Yamamoto, T. N., Patel, S. J., et al. (2016). Ionic immune suppression within the tumour microenvironment limits T cell effector function. Nature 2016 537:7621 537, 539-543.
Galletti, G., de Simone, G., Mazza, E. M. C., Puccio, S., Mezzanotte, C., Bi, T. M., Davydov, A. N., Metsger, M., Scamardella, E., Alvisi, G., et al. (2020). Two subsets of stem-like CD8+ memory T cell progenitors with distinct fate commitments in humans. Nature Immunology 2020 21:12 21, 1552-1562.
Gattinoni, L., Lugli, E., Ji, Y., Pos, Z., Paulos, C. M., Quigley, M. F., Almeida, J. R., Gostick, E., Yu, Z., Carpenito, C., et al. (2011). A human memory T cell subset with stem cell-like properties. Nat Med 17, 1290-1297.
He, W., Xu, J., Mu, R., Li, Q., Lv, D. Iun, Huang, Z., Zhang, J., Wang, C., and Dong, L. (2020). High-salt diet inhibits tumour growth in mice via regulating myeloid-derived suppressor cell differentiation. Nature Communications 2020 11:1 11, 1-17.
Jörg, S., Jan Kissel, Arndt Manzel, Markus Kleinewietfeld, Aiden Haghikia, Ralf Gold, Dominik N Müller, Ralf A Linker (2016) High salt drives Th17 responses in experimental autoimmune encephalomyelitis without impacting myeloid dendritic cells. Exp Neurol. 2016 May; 279:212-222. McLane, L. M., Abdel-Hakeem, M. S., and Wherry, E. J. (2019). CD8 T Cell Exhaustion During Chronic Viral Infection and Cancer. Annu Rev Immunol 37, 457-495.
Rayman, P., Uzzo, R. G., Kolenko, V., Bloom, T., Cathcart, M. K. Molto, L., Novick, A. C., Bukowski, R. M., Hamilton, T., Finke, JK. Tumor-induced dysfunction in interleukin-2 production and interleukin-2 receptor signaling: a mechanism of immune escape (2000). Cancer J Sci Am. 2000 February; 6 Suppl 1:S81-7.
Siddiqui, I., Schaeuble, K., Chennupati, V., Fuertes Marraco, S. A., Calderon-Copete, S., Pais Ferreira, D., Carmona, S. J., Scarpellino, L., Gfeller, D., Pradervand, S., et al. (2019). Intratumoral Tcf1+PD-1+CD8+ T Cells with Stem-like Properties Promote Tumor Control in Response to Vaccination and Checkpoint Blockade Immunotherapy. Immunity 50, 195-211.e10. https://doi.org/10.1016/J.IMMUNI.2018.12.021.

1. An in vitro or ex vivo method for obtaining an isolated population of CD8+ T cells comprising exposing isolated CD8+ T cells to an excess of sodium chloride by culturing said cells in a medium containing sodium chloride in a concentration of between 160 and 250 mM.

2. The method according to claim 1, wherein said sodium chloride concentration is between 160 and 180 mM.

3. The method according to claim 1 wherein said sodium chloride concentration is about 163.4 mM or about 183.4 mM.

4. The method according to claim 1 further comprising exposing said CD8+ T cells, before, simultaneously or after exposure to sodium chloride, to stimulators of activation and/or expansion of T cells.

5. The method according to claim 4 wherein exposure of said CD8+ T cells to stimulators of activation and/or expansion of T cells occurs by culturing said cells in a medium containing IL-2 and/or anti-CD3 and anti-CD28 antibodies conjugated to beads.

6. The method according to claim 1, wherein said isolated CD8+ T cells have been previously isolated from a subject.

7. The method according to claim 6 wherein said subject is a human subject.

8. The method according to claim 1 wherein said isolated CD8+ T cells are antigen-specific.

9. The method according to claim 1 further comprising:
a) plating CD8+T cells previously isolated from a subject in a suitable plate; and
b) adding to the plated cells a suitable medium comprising sodium chloride in a concentration of between 160 and 250 mM.

10. The isolated population of CD8+ T cells obtained by the method of claim 1.

11. The isolated population of CD8+ T cells according to claim 10, wherein said cells are characterized by expression of CD45RO, lack of expression of CCR7, an higher expression of IFNγ as compared to control CD8+ cells and an higher expression of marker CD107a as compared to control CD8+ cells.

12. A pharmaceutical composition comprising the isolated population of CD8+ T cells of claim 10 and at least one pharmaceutically acceptable carrier and/or vehicle.

13. (canceled)

14. A method for the prevention and/or treatment of cancer, comprising administering the isolated population of CD8+ T cells of claim 10 to a subject in need thereof wherein said CD8+ T cells are optionally autologous to the subject to be treated.

15. The method of claim 14 wherein said cancer is melanoma.

16. (canceled)

17. A method of adoptive cell therapy treatment in a subject, comprising:
exposing CD8+ T cells previously isolated from a subject to an excess of sodium chloride according to the method of claim 1; and
administering the obtained isolated CD8+ T cell population to a subject in need thereof, wherein said subject is optionally the same subject from which CD8+ T cells were previously isolated.

18. The method of claim 17 wherein said adoptive cell therapy is selected from Tumor-Infiltrating Lymphocyte (TIL) Therapy, Engineered T Cell Receptor (TCR) Therapy, Chimeric Antigen Receptor (CAR) T Cell Therapy and Natural Killer (NK) Cell Therapy.
